(19) United States
(12) Patent Application Publication
(10) Pub. No.: US 2007/0129434 A1
(43) Pub. Date: Jun. 7, 2007

(54) Analgesics and methods of use

(76) Inventors: Richard Smith-Carliss, West Chester, PA (US); Frank S. Caruso, Colts Necks, NJ (US); Peter A. Crooks, Nicholasville, KY (US); Kenneth J. Kellar, Bethesda, MD (US); Yingxian Xiao, Potomac, MD (US)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 10/501,692

(22) Filed: Jul. 15, 2004

Related U.S. Application Data
(63) Continuation of application No. PCT/US02/27936, filed on Aug. 29, 2002.

(51) Int. Cl. A61K 31/24

(52) U.S. Cl. 514/534

(57) ABSTRACT

A method for inducing analgesia and/or inhibiting abuse of abusive substances includes administration of d-methadone metabolites or their structural analogs. The d-methadone metabolites, EMDP and EDDP, and their structural analogs may be incorporated into a suitable pharmaceutical composition for administration to patients. The invention includes the method itself, certain structural analogs, and pharmaceutical compositions for use in accordance with the method.

### CROSS-REFERENCE TO RELATED APPLICATIONS

This application claims benefit of priority to U.S. provisional application Ser. No. 60/315,530 filed on Aug. 29, 2001, which is hereby incorporated by reference in its entirety.

### FIELD OF INVENTION

The invention relates to d-methadone metabolites and their analogs, as well as to methods of their use to induce analgesia and/or to inhibit abuse of abusive substances such as opioids, cocaine, nicotine, etc.

### DESCRIPTION OF THE RELATED ART

The study of pain and pain alleviation has made it clear that the development of pain alleviation is not a singular path. Many, varied sources of pain and its alleviation are known and suspected. For this reason, scientists continually search for more, different, and better ways of treating pain and of reducing side effects associated therewith.

Nicotinic acetylcholine receptors are distributed throughout the central and peripheral nervous systems where they mediate the actions of endogenous acetylcholine, as well as nicotine and other nicotinic agonists. They are often associated with cell bodies and axons of major neurotransmitter systems, and nicotinic agonists are thought to act through these receptors to promote the release of a number of neurotransmitters such as dopamine, norepinephrine, γ-aminobutyric acid, acetylcholine, and glutamate (for review, see Wonnacott, 1997), as well as certain pituitary hormones (Andersson et al., 1983; Sharp et al., 1987; Flores et al., 1989; Hulihian-Giblin et al., 1990). The release of this wide array of neurotransmitters and hormones probably contributes to the diverse, and sometimes opposite, effects of nicotine. For example, the release of norepinephrine is usually associated with arousal, while the stimulation of γ-aminobutyric acid systems is associated with sedation.

Nicotine was first examined for its potential as an analgesic drug almost 70 years ago (Davis et al., 1932), but its dose-response relationship for analgesia yielded a poor therapeutic index, which did not favor its development. More recently, following the discovery of the analgesic properties of epibatidine, a potent nicotinic agonist isolated from the skin of an Ecuadorian frog by Daly and colleagues (Spande et al., 1992), there has been renewed interest in the analgesic potential of drugs that act at nicotinic receptors (Bannon et al., 1998; Flores and Hargreaves, 1998; Flores, 2000).

It is likely that more than one neurotransmitter system plays an important role in analgesia. For example, methadone, a synthetic μ-opioid agonist, has analgesic properties similar to those of morphine (Kristensen et al., 1995), and it is also useful in the treatment of opiate addiction. Most of the morphine-like analgesic properties of (O)-methadone are as ascribed to the (−)-enantiomer, since the (+)-enantiomer has much weaker opiate properties (Scott et al., 1948; Smits and Myers. 1974; Horng et al., 1976). However, (+)-methadone does show analgesic potency in some experimental models (Shimoyama et al., 1997; Davis and Inturrisi, 1999), and it also appears to attenuate development of morphine tolerance (Davis and Inturrisi, 1999).

In addition to its agonist action at opiate receptors, methadone competes for [³H]MK801 binding sites within the NMDA receptor channel and blocks NMDA receptor-mediated responses (Ebert et al., 1995); furthermore, the two enantiomers of methadone are nearly equipotent at [³H]MK801 binding sites (Gorman et al., 1997). Several drugs such as MK801, phencyclidine, dextromethorphan, and dextrorphan, that block NMDA receptors, also block neuronal nicotinic receptors (Ramoa et al., 1990; Amador and Dani, 1991; Hernandez et al., 2000). Both nicotinic receptors and NMDA receptors have been implicated in pain pathways and possible mechanisms underlying the perception of pain. Therefore, the inventors examined the effects of methadone, its metabolites, and structural analogs (FIG. 1) on neuronal nicotinic receptors.

In addition to being involved in pain alleviation, recently, it has been discovered that certain nicotinic receptors may play a role in limiting abusive behavior.

Substances which may be subject to abuse include opioids, methamphetamines, hallucinogens, psychotropics, cocaine, and others. Some abusive substances are subtle and pervasive. Perhaps one of the most pervasive is nicotine, found in tobacco products. The term “abusive substances,” as used herein, refers to any substance that can lead to abuse by creating dependence or otherwise inducing drug-seeking behavior.

During their research into d-methadone and its metabolites, EMDP and EDDP, the inventors discovered that the EMDP and EDDP and novel analogs thereof induce analgesia and may be useful in independently or simultaneously deterring abuse of one or more abusive substances listed above.

### SUMMARY OF THE INVENTION

A method for inducing analgesia and/or inhibiting abuse of abusive substances includes administration of EMDP, EDDP, and novel analogs thereof. The compounds of the present invention may be incorporated into a suitable pharmaceutical composition for administration to patients. The invention includes novel compounds, a method for inducing analgesia and/or inhibiting abuse of an abusive substance, and pharmaceutical compositions for use in the method.

### BRIEF DESCRIPTION OF THE DRAWINGS

FIG. 1 depicts the chemical structures of methadone, EMDP, EDDP, analogs, and mecamylamine.

FIG. 2 is a graph depicting the effects of methadone versus nicotine on ⁸⁶Rb⁺ efflux from KXα3β4R2 cells.

FIG. 3 is a graph depicting the inhibition of nicotine-stimulated 8⁶Rb⁺ efflux; from KXα3β4R2 cells by methadone and its two enantiomers.

FIG. 4 is a graph depicting the competition by methadone for [³H]EB binding sites in membrane homogenates from KXα3β4R2 cells.

FIG. 5 is a graph depicting the noncompetitive inhibition of nicotine-stimulated ⁸⁶Rb⁺ efflux from KXα3β4R2 cells by methadone.

FIG. 6 is a graph depicting the comparison of the inhibition of nicotine-stimulated ⁸⁶Rb⁺ efflux from KXα3β4R2 cells by methadone, (+)-EDDP, LAAM, and mecamylamine.

FIG. 7 is a graph depicting the noncompetitive inhibition of nicotine-stimulated ⁸⁶Rb⁺ efflux from KXα3β4R2 cells by (+)-EDDP and LAAM.

FIG. 8 is a schematic of a synthesis reaction scheme for making various compounds in accordance with the invention.

FIG. 9 is another schematic of a synthesis reaction scheme for making various compounds in accordance with the invention.

FIG. 10 is a graph showing the analgesic effect of EDDP.

FIG. 11 depicts sample current inhibition by EDDP.

FIG. 12 depicts a concentration response curve.

FIG. 13 is a graph comparing the Glutamate stimulated Catecholamine release with treatment with MK-801, d-methadone, and R(+)EDDP in the hippocampus.

FIG. 14 is a graph comparing the Glutamate stimulated Catecholamine release with treatment with MK-801, d-methadone, and R(+)EDDP in the striatum.

### DETAILED DESCRIPTION

Definitions

Throughout this specification, reference simply to “the metabolites” or “d-methadone metabolites,” means EDDP and EMDP, as defined below, and the pharmaceutically acceptable salts thereof, unless otherwise indicated.

The term “(+)-methadone” means S-(+)-methadone hydrochloride;

the term “(−)-methadone” means R-(−)-methadone hydrochloride;

the term “LAAM” means (−)-α-acetylmethadol hydrochloride;

the term “(+)-EDDP” means R-(+)-2-ethyl-1,5-dimethyl-3,3-diphenylpyrrolinium perchlorate;

the term “(−)-EDDP” means S-(−)-2-ethyl-1,5-dimethyl-3,3-diphenylpyrrolinium perchlorate;

the term “(+)-EMDP” means R-(+)-2-ethyl-5-methyl-3,3-diphenyl-1-pyrroline hydrochloride;

the term “(−)-EMDP” means S-(−)-2-ethyl-5-methyl-3,3-diphenyl-1-pyrroline hydrochloride;

the term “EMDP” means (+)-EMDP, (−)-EMDP, or mixtures thereof;

the term “EDDP” means (+)-EDDP, (−)-EDDP, or mixtures thereof.

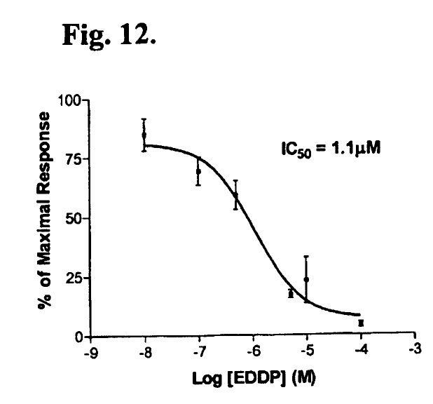

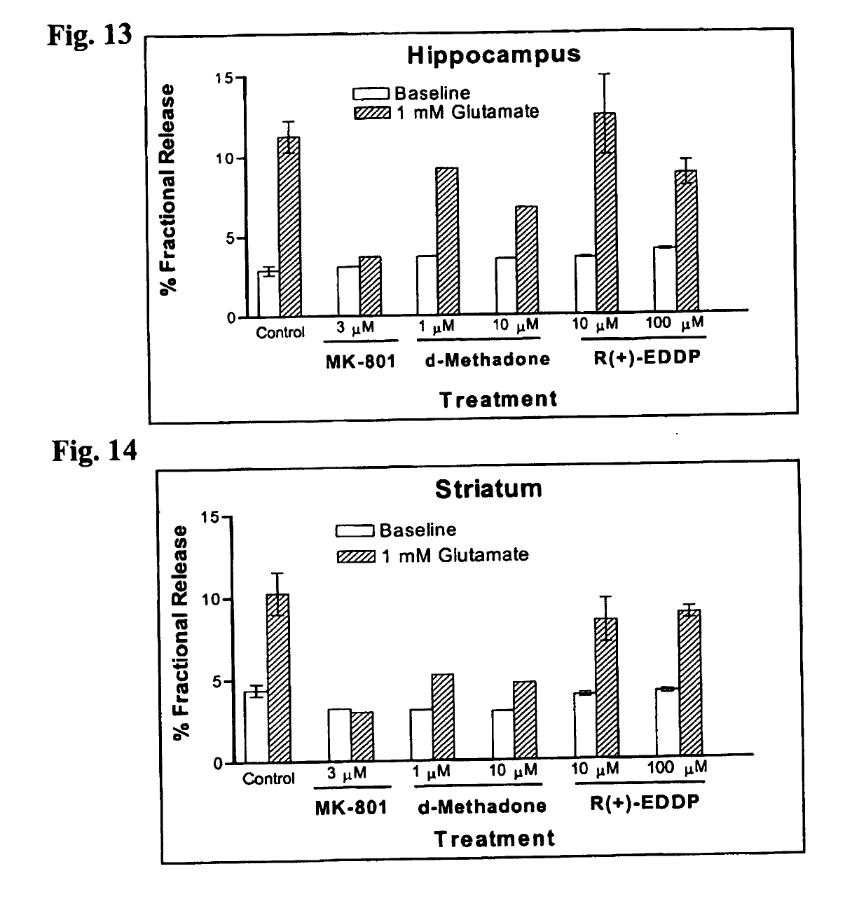

Despite the structural similarity to d-methadone, EMDP and EDDP, and analogs thereof, have different properties from d-methadone. FIGS. 13 and 14 demonstrate this by comparing the effect of MK-801, d-methadone and (+)-EDDP on glutamate stimulated catecholamine release in rat brain slices from the hippocampus and striatum. The hippocampus and striatum are both important and well-studied anatomical areas of the brain. The hippocampus is associated with learning and memory functions while the striatus is linked to motor function. Slices were loaded with [³H]norepinephrine or [³H]dopamine and then exposed to 1 mM glutamate for 2 min in the absence or presence of MK-801, d-methadone or (+)-EDDP. The baseline release was measured in the absence of glutamate. These results indicate, that (+)-EDDP is physiologically different from d-methadone, an opioid blocker, and MK-801, an NMDA blocker. This difference is apparent from the dose shift to the right, as seen in both FIGS. 13 and 14. Just 10 μM of d-methadone or MK-801 achieves partial block of catecholamine release while no effect is seen from (+)-EDDP until 100 μM.

The inventors believe, without being limited to this theory, that the success of the compounds of the present invention in inducing analgesia and/or inhibiting abuse is in their ability to block the nicotinic receptors. It should be noted, however, that binding or blocking of other sites may also contribute to the effect.

The action of d-methadone and the compounds of the present invention at α3β4 neuronal nicotinic receptors stably expressed in human embryonic kidney 293 cells was measured. These compounds are potent nicotinic receptor blockers. One of the compounds disclosed herein is among the most potent nicotinic receptor blockers that have been reported.

### Effects of Methadone and Related Drugs on nAChRs Experimental Procedures

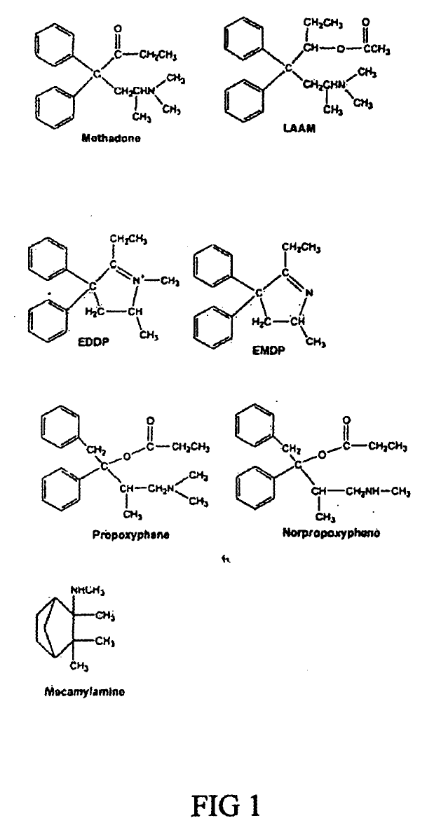

Materials and Drugs. Tissue culture medium, antibiotics, and serum were obtained from Invitrogen (Carlsbad, Calif.). [³H](±)-epibatidine and [⁸⁶Rb]rubidium chloride (⁸⁶Rb⁺) were obtained from PerkinElmer Life Science Products (Boston, Mass.). All other chemicals were purchased from Sigma Chemical Co. (St. Louis, Mo.) unless otherwise stated. (O)-Methadone hydrochloride (methadone), S-(+)-methadone hydrochloride [(+)-methadone], and R-(−)-methadone hydrochloride [(−)-methadone] were obtained from Sigma/RBI (Natick, Mass.). The following compounds were obtained from Research Triangle Institute (Research Triangle Park, N.C.) through the National Institute on Drug Abuse: (−)-α-acetylmethadol hydrochloride (LAAM, a methadone analog); R-(+)-2-ethyl-1,5-dimethyl-3,3-diphenylpyrrolinium perchlorate [(+)-EDDP, a methadone metabolite]; S-(−)-2-ethyl-1,5-dimethyl-3,3-diphenylpyrrolinium perchlorate [(−)-EDDP, a methadone metabolite]; R-(+)-2-ethyl-5-methyl-3,3-diphenyl-1-pyrroline hydrochloride [(+)-EMDP, a methadone metabolite]; S-(−)-2-ethyl-5-methyl-3,3-diphenyl-1-pyrroline hydrochloride [(−)-EMDP, a methadone metabolite]; (+)-α-propoxyphene hydrochloride (a methadone analog); and (+)-α-N-norpropoxyphene maleate (a propoxyphene metabolite). The structures of methadone, EMDP, EDDP, and several analogs used here are shown in FIG. 1, along with mecamylamine, a well-known nicotinic channel blocker.

Cell Culture. The cell line KXα3β4R2 was established previously by stably cotransfecting human embryonic kidney-293 cells with the rat α3 and β4 nAChR subunits genes (Xiao et al., 1998). Cells were maintained in minimum essential medium supplemented with 10% fetal bovine serum, 100 units/ml penicillin G, 100 mg/ml streptomycin, and 0.7 mg/ml of geneticin (G418) at 37° C. with 5% CO₂ in a humidified incubator.

⁸⁶Rb⁺ Efflux Assay. Function of nAChRs expressed in the transfected cells was measured using a ⁸⁶Rb⁺ efflux assay as described previously (Xiao et al., 1998). In brief, cells in the selection growth medium were plated into 24-well plates coated with poly(D-lysine). The plated cells were grown at 37° C. for 18 to 24 h to reach 70 to 95% confluence. The cells were then incubated in growth medium (0.5 ml/well) containing ⁸⁶Rb⁺ (2 μCi/ml) for 4 h at 37° C. The loading mixture was then aspirated and the cells were washed three times with buffer (15 mM HEPES, 140 mM NaCl, 2 mM KCl, 1 mM MgSO₄, 1.8 mM CaCl, 11 mM glucose, pH 7.4; 1 ml/well) for 30 s, 5 min, and 30 s, respectively. One milliliter of buffer, with or without compounds to be tested, was then added to each well. After incubation for 2 min, the assay buffer was collected for measurements of ⁸⁶Rb⁺ released from the cells. Cells were then lysed by adding 1 ml of 100 mM NaOH to each well, and the lysate was collected for determination of the amount of ⁸⁶Rb⁺ that was in the cells at the end of the efflux assay. Radioactivity of assay samples and lysates was measured by liquid scintillation counting. Total loading (cpm) was calculated as the sum of the assay sample and the lysate of each well. The amount of ⁸⁶Rb⁺ efflux was expressed as a percentage of ⁸⁶Rb⁺ loaded. Stimulated ⁸⁶Rb⁺ efflux was defined as the difference between efflux in the presence and absence of nicotine.

Experiments with antagonists were done in two different ways. For obtaining an IC₅₀ value, inhibition curves were constructed in which different concentrations of an antagonist were included in the assay to inhibit efflux stimulated by 100 mM nicotine. For determination of the mechanism of antagonist blockade, concentration-response curves for receptor activation by nicotine were constructed in the presence or absence of an antagonist. The maximal nicotine stimulated ⁸⁶Rb⁺ efflux (Eₘₐₓ) was defined as the difference between maximal efflux in the presence of nicotine and basal efflux. EC₅₀, Eₘₐₓ, and IC₅₀, values were determined by nonlinear least-squares regression analyses (GraphPad, San Diego, Calif.).

Ligand Binding Studies. The ability of compounds to compete for the agonist recognition site of nAChRs was determined in ligand binding studies as described previously (Houghtling et al., 1995; Xiao et al., 1998). Briefly, membrane preparations were incubated with [³H]EB for 4 h at 24° C. Bound and free ligands were separated by vacuum filtration through Whatman GF/C filters treated with 0.5% polyethylenimine. The radioactivity retained on the filters was measured by liquid scintillation counting. Total binding and nonspecific binding were determined in the absence and presence of (−)-nicotine (300 μM) respectively. Specific binding was defined as the difference between total binding and nonspecific binding. Binding curves were generated by incubating a series of concentrations of each compound with a single concentration of [³H]EB. The IC₅₀ and Kᵢ values of binding inhibition curves were determined by nonlinear least squares regression analyses.

### Results

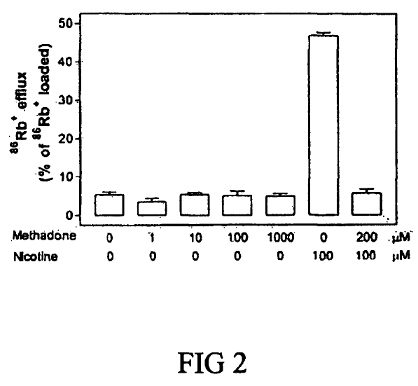

Effects of Methadone on ⁸⁶Rb⁺ Efflux from KXα3β4R2 Cells. FIG. 2. Effects of methadone versus nicotine on ⁸⁶Rb⁺ efflux from KXα3β4R2 cells. ⁸⁶Rb⁺ efflux as measured as described under Experimental Procedures. Cells were loaded with ⁸⁶ Rb⁺ and then exposed for 2 min to buffer alone (to measure basal release), or buffer containing methadone at the concentration, shown. 100 μM nicotine or 100 μM nicotine plus 200 μM methadone. The ⁸⁶Rb+efflux was response was expressed as a percentage of ⁸⁶Rb⁺ loaded. Data shown in FIG. 2 are the mean±standard error of four independent determinations. As shown in FIG. 2, at concentrations up to 1 mM, methadone did not increase ⁸⁶Rb⁺ efflux from KXα3β4R2 cells. In parallel assays, however, 100 μM nicotine stimulated ⁸⁶Rb⁺ efflux approximately 10-fold over basal levels, and this stimulation was completely blocked by 200 μM methadone. Thus demonstrating the blocking of α3β4 by methadone.

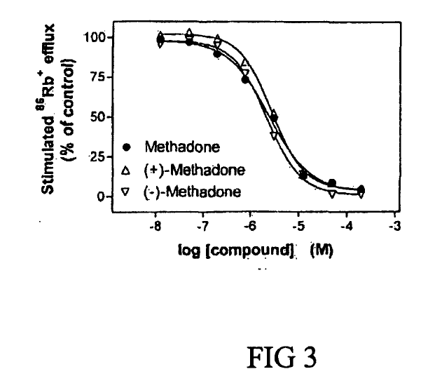

Potency of Methadone and Its Enantiomers in Inhibiting Nicotine-Stimulated ⁸⁶Rb⁺ Efflux from KXα3β4R2 Cells. The potencies of racemic methadone and its enantiomers as antagonists of the nAChRs were examined by measuring ⁸⁶Rb⁺ efflux stimulated by 100 μM nicotine in the presence of increasing concentrations of the compounds. Cells were loaded with and then exposed for 2 min to buffer alone (basal release) or buffer containing 100 μM nicotine in the absence or presence of racemic methadone or one of the methadone enantiomers at the concentrations shown. ⁸⁶Rb⁺ efflux was expressed as a percentage of ⁸⁶Rb⁺ loaded, and control values were defined as ⁸⁶Rb⁺ efflux stimulated by 100 μM nicotine in the absence of methadone. Inhibition curves shown in FIG. 3 are from a single experiment measured in quadruplicate. See Table 1 for mean and standard error of the IC₅₀ values. As illustrated in FIG. 3, racemic methadone potently inhibited nicotine-stimulated ⁸⁶Rb⁺ efflux in a concentration-dependent manner with an IC₅₀ of approximately 2 μM. Moreover, (+)-methadone and (−)-methadone inhibited the, function of these receptors with similar potencies (FIG. 3; Table 1).

TABLE 1 lists the inhibitory properties of enantiomers of methadone and compounds of the present invention on nicotine-stimulated ⁸⁶Rb⁺ efflux from KXα3β4R2 cells. IC₅₀ values were calculated front inhibition curves in which ⁸⁶Rb⁺ efflux was stimulated by 100 μM nicotine, as described under Experimental Procedures. Mecamylamine, a standard nAChR antagonist, was included for comparison. Data shown are the mean±standard error of three to six independent measurements.

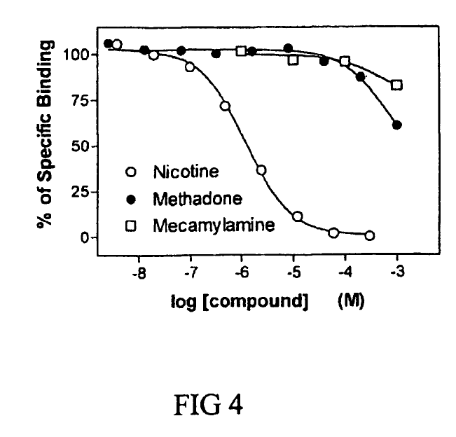

Low Affinities of Methadone for nAChR Agonist Binding Sites. The ability of methadone to compete for α3β4 receptor agonist recognition sites labeled by [³H]EB in membranes from KXα3β4R2 cells was examined. Binding assays were carried out as described under Experimental Procedures using 323 μM [³H]EB. The K; value for nicotine was 559 nM. The Kᵢ values for methadone and mecamylamine cannot be estimated because there was less than 50% inhibition even at the highest concentration used (1 mM). As shown in FIG. 4 methadone does not compete effectively for [³H]EB binding sites. Mecamylamine is shown for comparison. Thus, even at the highest concentration used (1 mM), methadone inhibited less than 50% of [³H]EB binding to α3β4 receptors. This was comparable to the weak binding potency of mecamylamine. In parallel assays carried out as positive controls, nicotine competed effectively for the agonist binding sites of α3β4 receptors, yielding a dissociation constant (Kᵢ) of 560 nM, which is similar to that previously reported in these cells (Xiao et al., 1998). Methadone's very low affinity for the agonist recognition sites of α3β4 receptors contrasts with its high potency in blocking receptor function (IC₅₀ of about 2 μM) and suggests a noncompetitive mechanism of receptor antagonism.

TABLE 1

| Drug | IC₅₀ |
|---|---|
| (+)-Mehtadone | μM |
| (−)-Methadone | 1.9 ± 0.2 |
| (+)-Methadone | 2.5 ± 0.2 |
| (−)-Methadone | 2.0 ± 0.3 |
| (+)-EDDP | 0.4 ± 0.2 |
| (−)-EDDP^{a} | 0.4 ± 0.1^{a} |
| (+)-EMDP | 5.8 ± 1.0 |
| (−)-EMDP | 6.3 ± 0.7 |
| Propoxyphene | 2.7 ± 0.4 |
| Norpropoxyphene | 1.8 ± 0.1 |
| LAAM | 2.5 ± 0.4 |
| Mecamylamine | 1.1 ± 0.2 |
| Dextromethorphan | 8.9 ± 1.1 |
| Dextrorphan | 29.6 ± 5.7 |
| Mecamylamine | 1.0 ± 0.04 |
| MK-801 | 26.6 ± 9.6 |

^{a}The IC₅₀ value for (−)-EDDP significantly lower than that for mecamylamine (p < 0.02).

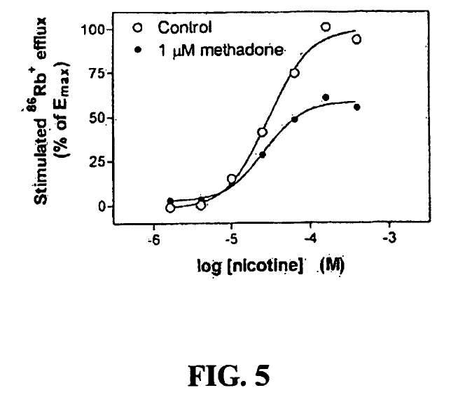

Noncompetitive Block of nAChR Function by Methadone. To definitively identify the type of receptor blockade by methadone, we examined its effect on concentration-response curves for receptor activation by nicotine. ⁸⁶Rb⁺ efflux was measured as described under Experimental Procedures. Cells were loaded with ⁸⁶Rb⁺ and then exposed to buffer containing increasing concentrations of nicotine for 2 min in the absence (control) or presence of 1 μM methadone. The ⁸⁶Rb⁺ efflux was calculated as a percentage of ⁸⁶Rb⁺ loaded, and the Eₘₐₓ was defined as the maximum response in the absence of methadone. The curves shown are from a single experiment measured in quadruplicate. The EC₅₀ Values in the absence and presence of methadone were 28.8±1.2 and 21.3±2.1 μM, respectively (mean±standard error from four independent experiments). The Eₘₐₓ, value (mean±standard error) in the presence of 1 μM methadone was 63±2% of control values. Both the ECₘₐₓ (p<0.05) and Eₘₐₓ values (p<0.01) in the presence of methadone are, significantly different from control values As shown in FIG. 5, in the presence of 1 μM methadone, the maximum ⁸⁶Rb⁺ efflux stimulated by nicotine (Eₘₐₓ) was markedly reduced, but the EC₅₀ for nicotine was altered only slightly, if at all. This result indicates that methadone does, in fact, block α3β4 nAChR function primarily by a noncompetitive mechanism.

Inhibitory Effects of Methadone Metabolites and Structural Analogs on ⁸⁶Rb⁺ Efflux from KXα3β4R2 Cells. We tested seven compounds related to methadone, including its metabolites and structural analogs, for their agonist and antagonist effects on ⁸⁶Rb⁺ efflux from KXα3β4R2 cells At concentrations up to 100 μM, none of these compounds increased ⁸⁶Rb⁺ efflux (data not shown).

Effects of Methadone and Related Drugs on nAChRs

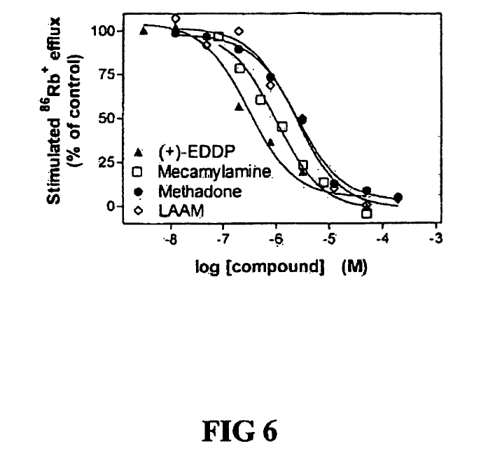

However, all of the compounds tested here were relatively potent blockers of nicotine-stimulated ⁸⁶Rb⁺ efflux (See Table 1). Thus, the long-acting methadone analog LAAM as well as propoxyphene and norpropoxyphene were about as potent as methadone in blocking this α3β4 receptor-mediated response. The methadone metabolite EDDP was even more potent; in fact, EDDP appears to be one of the most potent nAChR antagonists that has been reported, being about 5 times more potent than methadone and about twice as potent as mecamylamine (FIG. 6; Table 1). Furthermore, like methadone, the two enantiomers of the metabolites were equipotent in blocking α3β4 nAChR (Table 1), although in these studies the difference in IC₅₀ values between (−)-EDDP and mecamylamine was statistically significant (p<0.02), while that for (+)-EDDP was not (0.05<p<0.1).

FIG. 6 Shows the comparison of the inhibition of nicotine-stimulated ⁸⁶Rb⁺ efflux from KXα3β4R2 cells by methadone, (+)-EDDP, LAAM, and mecamylamine. ⁸⁶Rb⁺ efflux was measured as described under Experimental Procedures. Cells were loaded with ⁸⁶Rb⁺ and then exposed for 2 min to buffer alone (basal release) or buffer containing 100 μM nicotine in the absence or presence of racemic methadone, (+)-EDDP, LAAM, or mecamylamine at the concentrations shown. ⁸⁶Rb⁺ efflux was expressed as percentage of ⁸⁶Rb⁺ loaded and control values were defined as ⁸⁶Rb⁺ efflux stimulated by 100 μM nicotine in the absence of methadone.

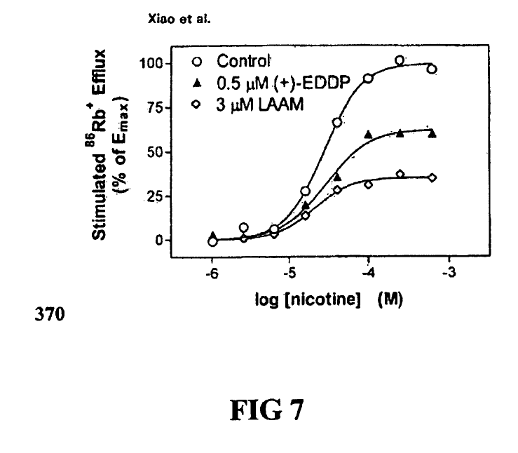

Noncompetitive Block of nAChR Function by Methadone Metabolites and Structural Analogs. None of the compounds examined here competed effectively for [³H]EB binding sites), suggesting that, like methadone, they block receptor function via a noncompetitive mechanism. To examine this more directly, the effects of (+)-EDDP and LAAM on concentration-response curves for receptor activation by nicotine were examined. ⁸⁶Rb⁺ efflux was measured as described under Experimental Procedure. Cells were loaded with ⁸⁶Rb⁺ and then exposed to buffer containing increasing concentrations of nicotine for 2 min in the absence (control) or presence of 0.5 μM EDDP of 3 μM LAAM. The ⁸⁶Rb⁺ efflux was calculated as a percentage of ⁸⁶Rb⁺ loaded, and the EC₅₀ was defined as the maximum response in the absence of antagonists. The curves shown are from a single experiment measured in quadruplicate. The EC₅₀ values for nicotine-stimulated ⁸⁶Rb⁺ efflux in the control cells, in the presence of 0.5 μM (+)EDDP, and in the presence of 3 μM LAAM were, respectively, 28.2±1.5, 25.5±1.5, and 18.8±1.4 μM*. The Eₘₐₓ, values in the presence of 0.5 μM (+)-EDDP and 3 μM LAAM were, respectively 60±3** and 44±5%** of control. Values are mean±standard error from three independent experiments. The values that were significantly different from values of control are indicated by *p<0.05 and **p<0.01, respectively. As shown in FIG. 7, both of these compounds acted as noncompetitive blockers of α3β4 nicotinic receptors.

### Discussion

We investigated the effects of the enantiomers of methadone and its metabolites as well as three structural analogs of methadone on the function of rat α3β4 nAChRs stably expressed in KXα3β4R2 cells. All of these compounds inhibited nicotine-stimulated ⁸⁶Rb⁺ efflux in a concentration-dependent manner and with relatively high potencies, comparable with that of mecamylamine. In particular, EDDP, the major oxidative metabolite of methadone, with an IC₅₀ of about 0.4 μM, is one of the most potent nicotinic antagonists that has been reported.

A noncompetitive mechanism of nAChR blockade by methadone, EDDP, and LAMM is clearly indicated by the marked decrease in the maximum receptor-mediated response without a substantial change in the EC₅₀ value for nicotine-stimulated ⁸⁶Rb⁺ efflux in the presence of these compounds. A noncompetitive mechanism is also consistent with the observation that neither methadone, its metabolites, nor its structural analogs compared effectively for [³H]EB binding sites, which represent the agonist recognition site of the receptor. Taken together, these data indicate that all of these compounds most likely block within the α3β4 nAChR channel. There also appeared to be a slight but statistically significant decrease in the EC₅₀ value for nicotine-stimulated ⁸⁶Rb⁺ efflux in the presence of methadone and LAAM, implying that these drugs might actually increase the potency of nicotine at the receptor. Although it is very probable that the small difference in nicotine's EC₅₀ values represents a statistical artifact, we cannot rule out an allosteric effect.

The (+)- and (−)-enantiomers of methadone and its metabolites are equipotent in blocking nAChR. This is in contrast to methadone's agonist actions at opiate receptors, which are ascribed almost entirely to its (−)-enantiomer. Therefore, the high potency of the (+)-enantiomers of methadone and its metabolites should allow blockade of nicotinic receptors without necessarily stimulating opiate receptors. This could then permit these (+)-enantiomers to be used in conditions where blockade of neuronal nicotinic receptors might be beneficial. For example, receptor blockade by mecamylamine is reported to aid in smoking cessation (Rose et al., 1994, 1998), and the most potent of the methadone metabolites is approximately twice as potent as mecamylamine. In addition, nicotinic receptors are thought to play a potentially important role in some analgesia pathways (Flores, 2000). Although analgesia has most often been associated with nicotinic agonists, these actions are incompletely understood, and it is possible that nicotinic antagonists can also contribute to analgesia (Hamann and Martin, 1992). If this were the case for methadone and its metabolites, their analgesic effect through nicotinic mechanisms would perhaps be additive to analgesia mechanisms mediated by opiate receptors. This would be particularly useful where tolerance to opiates and/or ceiling effects are issues. In fact, both dextromethorphan, which blocks NMDA and nicotinic receptors, and (+)-methadone are reported to attenuate the development of tolerance to morphine analgesia (Elliott et al., 1994; Davis and Inturrisi, 1999).

The plasma concentration of methadone following a single dose is approximately 0.25 μM (Inturrisi and Verebely, 1972) and the steady-state concentration in patients taking methadone chronically can exceed 1 μM (de Vos et al., 1995; Alburges et al., 1996; Dyer et al., 1999). At these concentrations, methadone could be expected to produce significant blockade of α3β4 nicotinic receptors. The steady-state plasma concentration of the more potent EDDP is usually much lower, but the peak concentration following administration of methadone can approach 0.2 μM (de Vos et al., 1995).

It should also be noted that (+)-methadone blocks NMDA receptor channels with potencies similar to, although slightly lower than, those found here at nicotinic receptors (Gorman et al., 1997; Stringer et al., 2000). Methadone's block of NMDA receptors also has been linked to its analgesic actions (Shimoyama et al., 1997; Davis and Inturrisi, 1999), and particularly to its potential usefulness for treating chronic and/or neuropathic pain (Elliott et al., 1995; Hewitt, 2000; Stringer et al., 2000). In addition, methadone's possible attenuation of morphine tolerance may involve NMDA receptors (Gorman et al., 1997; Davis and Inturrisi, 1999). In this regard, however, the block of nicotinic receptors by EDDP and (+)-methadone might also contribute directly to analgesic actions and even to the attenuation of morphine tolerance. Thus, it is possible that methadone and its metabolites can affect three different neurotransmission systems that have been associated with analgesia pathways and tolerance to opiates.

Accordingly, the compounds of the present invention block α3β4 nicotinic cholinergic receptors by a noncompetitive mechanism consistent with channel blockade. Both the (+)- and (−)-enantiomers of methadone and its metabolites are active; therefore, the high potency of the (+)-enantiomers of these compounds, particularly EDDP, in blocking nicotinic receptors should allow them to be used as probes of nicotinic receptors without affecting opiate receptors.

The Compounds

In describing the compounds, the following definitions are used, each of which includes all possible geometric, racemic, diasteriomeric, and enantiomeric forms thereof:

The term alkyl includes branched and straight chain, saturated and unsaturated, substituted and unsubstituted alkyl groups. Examples of alkyls include methyl, ethyl, propyl, isopropyl, butyl, tert-butyl, etc.

The term alkenyl refers to an ethylenically unsaturated hyrdocarbon group, straight or branched, which may be substituted or unsubstituted.

The term alkynyl refers to a straight or branched hydrocarbon group having 1 or 2 acetylenic bonds, which may be substituted or unsubstituted.

The term aryl refers to phenyl, which may be substituted with 1-5 substituents.

The term azaaromatic refers to an aromatic ring containing 1-3 nitrogen atoms, which may be substituted with 1-5 substituents.

The general structure of these compounds is set forth as Formulae I and II below, and include all possible geometric, racemic, diasteriomeric, and enantiomeric forms thereof:

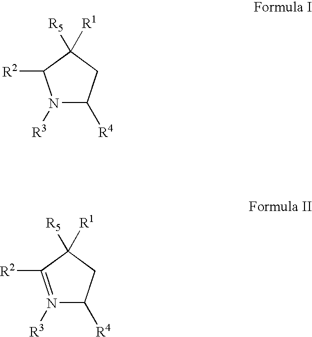

where:

R¹ is H, (C₁-C₆)alkyl, (C₃-C₆)cycloalkyl-(C₁-C₆)alkyl, (C₃-C₆)cycloalkyl-C₁-C₆)alkenyl, and aryl or azaaromatic having 1-5 substituents independently selected from the group consisting of hydrogen, (C₁-C₆)alkyl, (C₃-C₆)cycloalkyl, (C₂-C₆)alkenyl, aryl, and aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso;

R² is hydrogen, (C₁₋₆)alkyl, (C₂-C₆)alkene, or (C₂-C₆)alkynyl, and in Formula I, R² may also be selected from O═ or HN═;

R³ is selected from hydrogen, (C₁-C₆)alkyl, (C₃-C₆)cycloalkyl, (C₂-C₆) alkenyl, aryl, and aryl(C₁-C₆)alkyl;

Preferably, R³ is methyl or ethyl;

R⁴ is C₁-C₆ alkyl, and (C₃-C₆)cycloalkyl; and

R⁵ is aryl or azaaromatic having 1-5 substituents independently selected from the group consisting of hydrogen, (C₁-C₆)alkyl, (C₃-C₆)cycloalkyl, (C₂-C₆)alkenyl, aryl, and aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso and may form a bond to R¹ to result in a conjugated ring system.

The compounds may be in the form of pharmaceutically acceptable salts, including but not limited to inorganic acid addition salts such as hydrochloride, hydrobromide, sulfate, phosphate and nitrate; organic acid addition salts such as acetate, galactarate, propionate, succinate, lactate, glycolate, malate, tartrate, citrate, maleate, fumarate, methanesulfonate, salicylate, p-toluenesulfonate, benzenesulfonate, and ascorbate; salts with acidic amino acids such as aspartate and glutamate; the salts may in some cases by hydrates or solvates with alcohols and other solvents. Salt forms can be prepared by mixing the appropriate amine with the acid in a conventional solvent, with or without alcohols or water.

More specifically, the following compounds are contemplated:

| | | | | | | | Formula |
|---|---|---|---|---|---|---|---|
| Structure | X | R¹ | R² | R³ | R⁴ | R⁵ | Series |
| 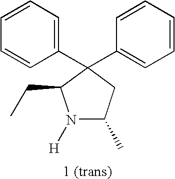 | C | phenyl | CH₂CH₃ | H | CH₃ | phenyl | I/1 |
| 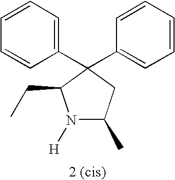 | C | phenyl | CH₂CH₃ | H | CH₃ | phenyl | I/1 |
| 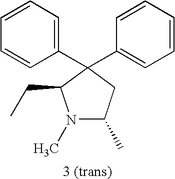 | C | phenyl | CH₂CH₃ | CH₃ | CH₃ | phenyl | I/1 |
| 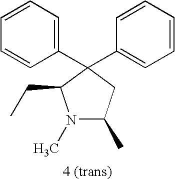 | C | phenyl | CH2CH₃ | CH₃ | CH₃ | phenyl | I/1 |
| 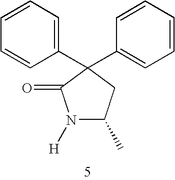 | C | phenyl | ═O | H | CH₃ | phenyl | I/1 |
| 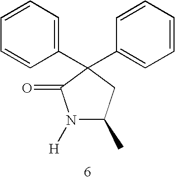 | C | phenyl | ═O | H | CH₃ | phenyl | I/1 |
| 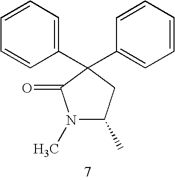 | C | phenyl | ═O | CH₃ | CH₃ | phenyl | I/1 |
| 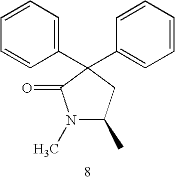 | C | phenyl | ═O | CH₃ | CH₃ | phenyl | I/1 |
| 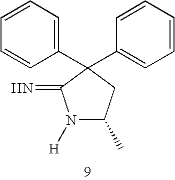 | C | phenyl | ═NH | H | CH₃ | phenyl | I/1 |
| 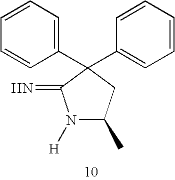 | C | phenyl | ═NH | H | CH₃ | phenyl | I/1 |
| 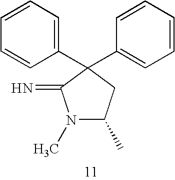 | C | phenyl | ═NCH₃ | H | CH₃ | phenyl | I/1 |
| 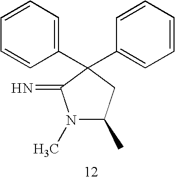 | C | phenyl | ═NCH₃ | H | CH₃ | phenyl | I/1 |
| 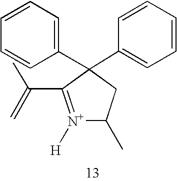 | C | phenyl | —CCH₃CH₂ | H | CH₃ | phenyl | II/1 |
| 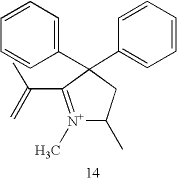 | C | phenyl | —CCH₃CH₂ | CH₃ | CH₃ | phenyl | II/1 |
| 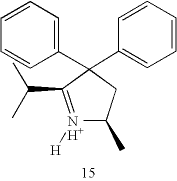 | C | phenyl | —CH(CH₃)₂ | H | CH₃ | phenyl | II/1 |
| 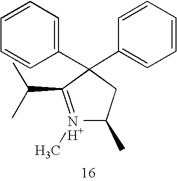 | C | phenyl | —CH(CH₃)₂ | CH₃ | CH₃ | phenyl | II/1 |
| 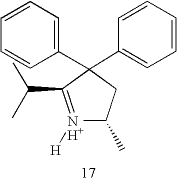 | C | phenyl | —CH(CH₃)₂ | H | CH₃ | phenyl | II/1 |
| 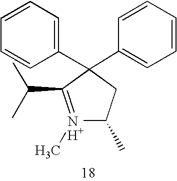 | C | phenyl | —CH(CH₃)₂ | CH₃ | CH₃ | phenyl | II/1 |
| 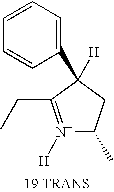 | C | H | —CH₂CH₃ | H | CH₃ | phenyl | II/2 |
| 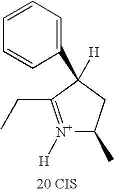 | C | H | —CH₂CH₃ | H | CH₃ | phenyl | II/2 |
| 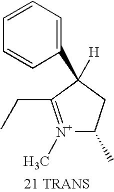 | C | H | —CH₂CH₃ | CH₃ | CH₃ | phenyl | II/2 |
| 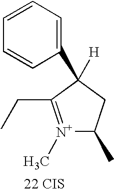 | C | H | —CH₂CH₃ | CH₃ | CH₃ | phenyl | II/2 |
| 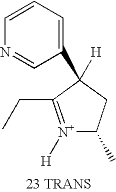 | N | H | —CH₂CH₃ | H | CH₃ | 3-pyridinyl | II/2 |
| 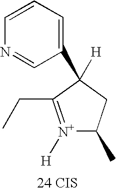 | N | H | —CH₂CH₃ | H | CH₃ | 3-pyridinyl | II/2 |
| 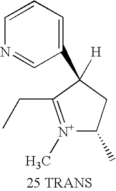 | N | H | —CH₂CH₃ | CH₃ | CH₃ | 3-pyridinyl | II/2 |
| 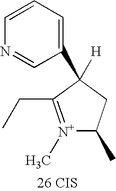 | N | H | —CH₂CH₃ | CH₃ | CH₃ | 3-pyridinyl | II/2 |
| 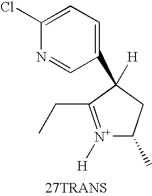 | N | H | —CH₂CH₃ | H | CH₃ | 4-chloro-3-pyridinyl | II/2 |
| 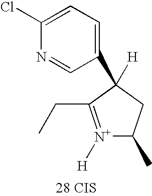 | N | H | —CH₂CH₃ | H | CH₃ | 4-chloro-3-pyridinyl | II/2 |
| 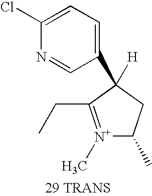 | N | H | —CH₂CH₃ | CH₃ | CH₃ | 4-chloro-3-pyridinyl | II/2 |
| 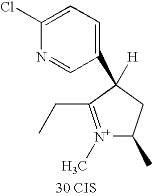 | N | H | —CH₂CH₃ | CH₃ | CH₃ | 4-chloro-3-pyridinyl | II/2 |
| 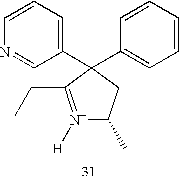 | N | phenyl | —CH₂CH₃ | H | CH₃ | pyridinyl | II/1 |
| 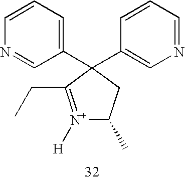 | N | pyridinyl | —CH₂CH₃ | H | CH₃ | pyridinyl | II/1 |
| 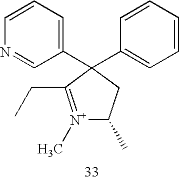 | N | phenyl | —CH₂CH₃ | CH₃ | CH₃ | pyridinyl | II/1 |
| 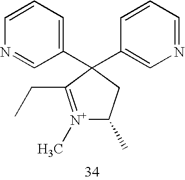 | N | pyridinyl | —CH₂CH₃ | CH₃ | CH₃ | pyridinyl | II/1 |
| 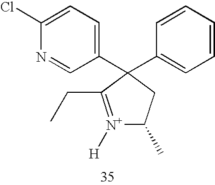 | N | phenyl | —CH₂CH₃ | H | CH₃ | 4-chloro-3-pyridinyl | II/1 |
| 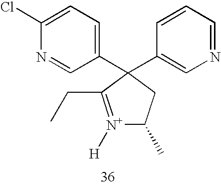 | N | pyridinyl | —CH₂CH₃ | H | CH₃ | 4-chloro-3-pyridinyl | II/1 |
| 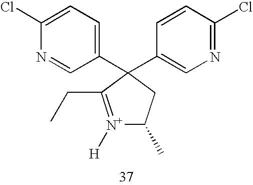 | N | 4-chloro-3-pyridinyl | —CH₂CH₃ | H | CH₃ | 4-chloro-3-pyridinyl | II/1 |
| 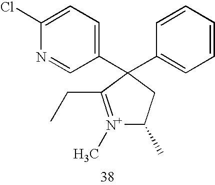 | N | phenyl | —CH₂CH₃ | CH₃ | CH₃ | 4-chloro-3-pyridinyl | II/1 |
| 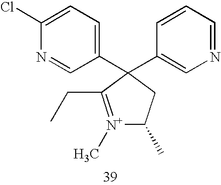 | N | pyridinyl | —CH₂CH₃ | CH₃ | CH₃ | 4-chloro-3-pyridinyl | II/1 |
| 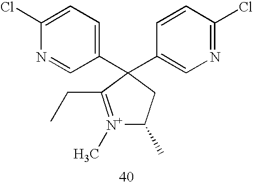 | N | 4-chloro-3-pyridinyl | —CH₂CH₃ | CH₃ | CH₃ | 4-chloro-3-pyridinyl | II/1 |

* = N indicates that there is a double bond in the five membered ring between R³ and the carbon carrying R².

Compounds where R⁵ bonds to R¹ such as those set forth below may also be used, and can be made through simple alterations to the synthesis of the above compounds.

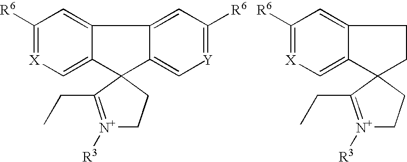

where

X and Y are independently selected from the group consisting of C and N;

R³ is as set forth above;

R⁶ is independently selected from the group consisting of hydrogen, (C₁-C₆)alkyl, (C₃-C₆)cycloalkyl, (C₂-C₆)alkenyl, aryl, and aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino.

Exemplary Syntheses

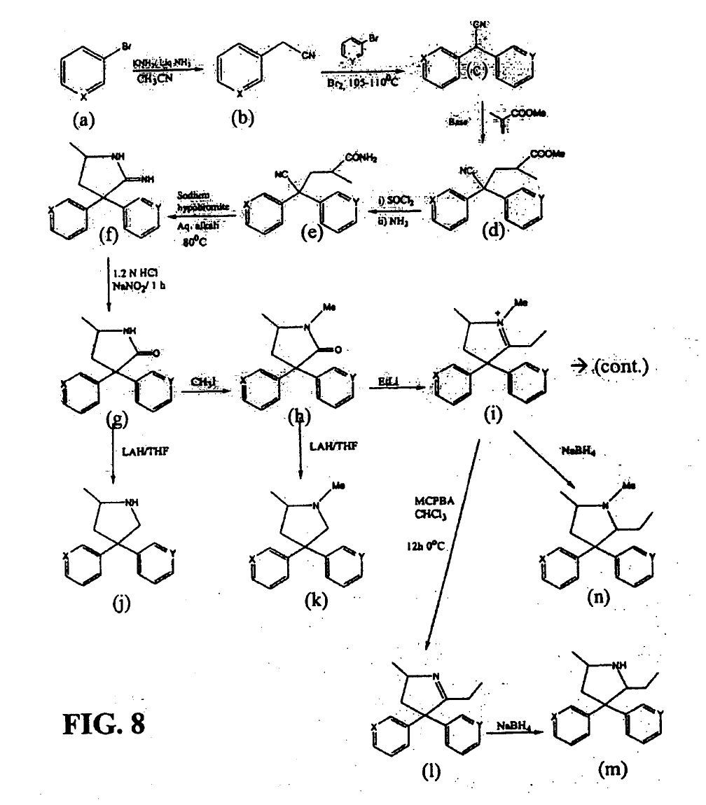

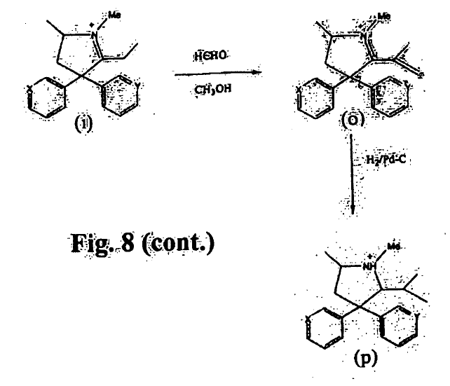

FIGS. 8 and 9 show some exemplary synthesis reactions that may be used to produce these compounds. The compounds disclosed in the syntheses include all possible geometric, racemic, diasteriomeric, and enantiomeric forms unless otherwise noted. Structures listed in parentheses correspond to those listed in the above table. Those skilled in the art will recognize that these compounds may be formed by other sythesis reactions, and that simple modifications to these syntheses will produce similar products, all of which are considered within the scope of this invention.

Series 1

FIG. 8 shows the basic synthesis reaction, which produces Compound (f) (Structures 9 and 10). First, bromobenzene (a), or bromoheterocycle where X is a heteroatom at any position, is mixed with CH₃CN and KNH₂ in liquid ammonia to yield (b). Which is then mixed with a second bromobenzene or heterocycle, where Y is a heteroatom selected independently of X at any location, with Br₂ at 105-110° C. to yield the diphenyl cyanide (c). This product is then reacted in a basic solution, with t-butylenemetioxylate to yield compound (d). Compound (d) is reacted with SOCl₂ and ammonia to produce compound (f), the amidino analogs. Those skilled in the art will recognize that, in light of this synthesis, compounds 11 and 12, and other variations, may be made simply by similar methods.

Synthesis of compounds (g) and (j)

The compound (f) is further reacted with 1.2N HCl with NaNO₂ for about 1 hour to yield a compound (g), (Structures 5 and 6). Reaction of this mixture with LAH/THF yields compound (j), which also may be used in the methods disclosed herein.

Synthesis of Compounds (h) and (k)

Beginning where the reaction left off with compound (g), above, further reaction with CH₃I substitutes a methyl group to the nitrogen of the five membered ring to yield compound (h) (Structures 7 and 8). Compound (k) is achieved by reacting this mixture with LAH/THF.

Synthesis of Compounds (i), (l), (m), (n), (O), and (p)

Picking up the reaction at the formation of compound (h), further reaction with EtLi to open the double bonded oxygen yields compound (i) (Structures 33 and 34). Compound (i) is then the basis for three other chains of reaction.

Compound (l) is formed by reacting compound (i) with MCPBA and CHCl₃ for 12 hours at 0° C. Compound (m) (Structures 1 and 2) is then formed by reacting this with NaBH₄.

Compound (n) (Structures 3 and 4) are produced by reacting compound (i) with NaBH₄.

Compound (i) is reacted with HCHO and CH₃OH to produce compound (O) (Structure 14), which is then reacted with H₂ and Pd—C to yield Compound (p) (Structures 16, 18).

Series 2

The synthesis reaction for series two is identical to that for series one except that the second step of mixing a second bromobenzene (b₂), or bromoheterocycle, is omitted. Similar mono-phenyl compounds are thus produced. FIG. 9 sets out the synthesis reaction for series two. Parrallel compounds to those of Series 1 are indicated with references characters with the subscript 2.

Analgesia and Abuse Deterrance

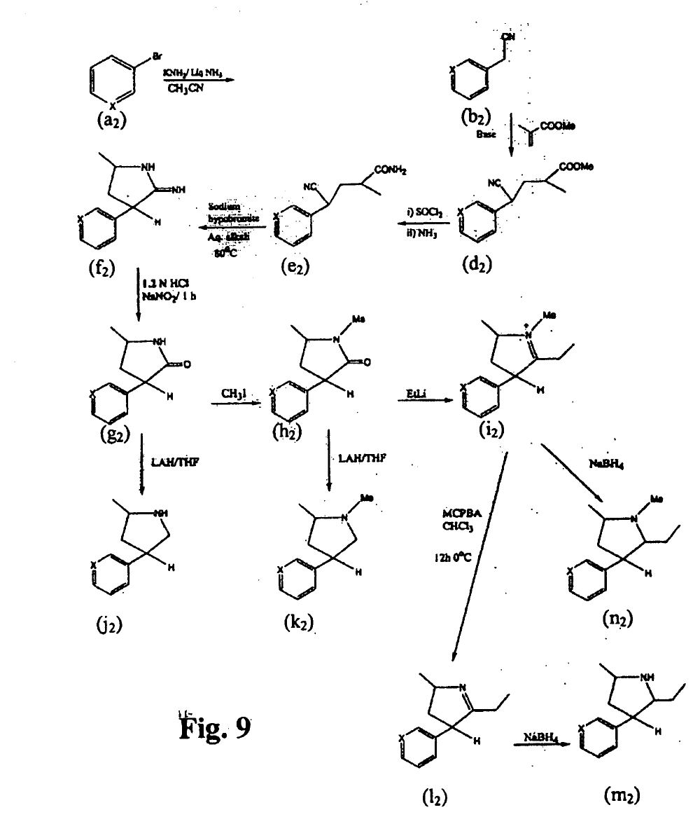

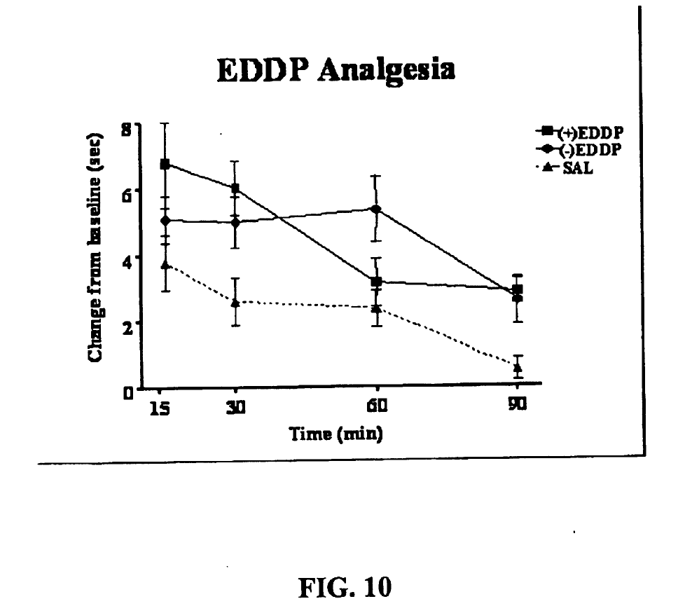

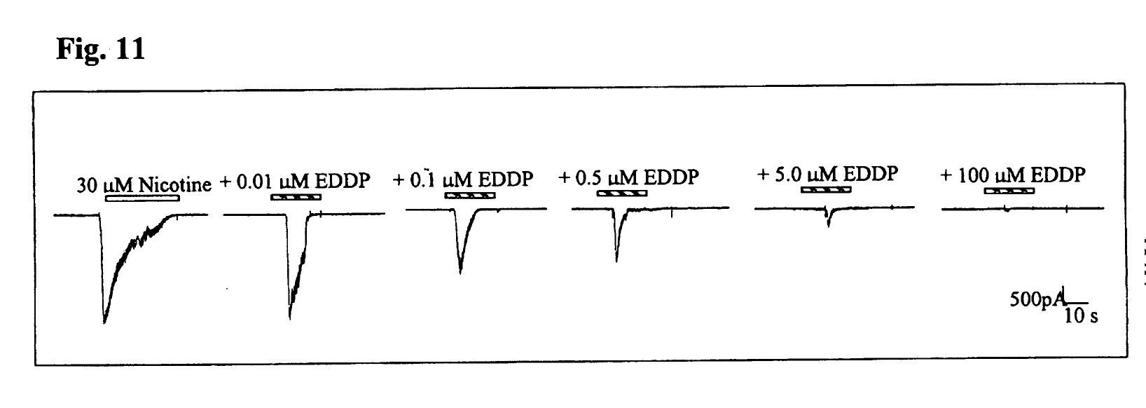

To confirm their suspicions that the compounds of the present invention, do in fact have an analgesic effect, the inventors experimented with mice. FIG. 10 shows the results of an experiment conducted on naive, adult, Swiss-Webster mice. Each enantiomer of EDDP, in 40 μg doses, was administered intracerebrally to the mice. The animals were monitored for baseline sensitivity using the warm-water tail-withdrawal nociception assay and the latency to tail withdrawal was monitored as a measurement of analgesia. The results demonstrate that tail withdrawal latency increased with the administration of either enantiomer of EDDP. Thus, it is clear that the d-methadone metabolite EDDP has significant analgesic effect. Likewise, the metabolite EMDP and the structural analogs of both EDDP and EMDP are expected to do the same. FIGS. 11 and 12 illustrate the effect of EDDP concentration on the inhibition of nicotine activated currents, which is one explanation for the analgesic effect.

As discussed in detail above, the inventors believe the d-methadone metabolites and their analogs block the nicotinic α3β4 receptor. Recently, it has been reported that dextromethorphan and dextrorphan, α3β4 blockers, actually deter abuse of abusive substances. Glick et al. report a decrease in self-administration of each of morphine, methamphetamine, and nicotine in rats when exposed to 5-30 mg/kg of these specific α3β4 blockers. Glick S D, Maisonneuve I M, Dickinson H A, Kitchen B A; Comparative effects of dextromethorphan and dextrorphan on morphine, methamphetamine, and nicotine self-administration in rats; Eur J Pharmacol. 2001 Jun. 22;422(1-3):87-90. Because of their discovery that the d-methadone metabolites and their structural analogs are α3β4 blockers, the current inventors contemplate that the d-methadone metabolites and their analogs also have such deterrent affects.

The inventors do not wish to be bound by this theory, but believe that the d-methadone metabolites or structural analogs interfere with the reward component of the abusive substance. The reward component is often thought of as the euphoric effect, as inducing drug seeking behavior. The administration of the d-methadone metabolites or structural analogs interferes with these effects, and deters abuse as a result. Such administration will aid in smoking cessation and deter abuse of more hard core substance.

Accordingly, administration of the d-methadone metabolites or their structural analogs can actually deter abuse of abusive substances from the opioids to nicotine.

Administration

The compounds of the present invention may be administered to patients in effective amounts or effective doses to alleviate pain and/or deter abuse of an abusive substance. In another embodiment, the compounds are administered in combination with abusive substances, particularly opioids or other analgesics, in a single pharmaceutical composition. In this scenario, the compounds of the present invention contribute to the analgesic effect while also deterring the abuse of the companion compound. Thus, patients benefit from the added analgesic effect of the compound, while gaining the added benefit of reduced potential for abuse. In another embodiment, the compounds of the present invention are administered independently of an abusive substance to induce analgesia. In yet another embodiment, the independent administration of the compounds serves to deter abuse of a separately administered abusive substance.

By “effective amount,” “therapeutic amount,” or “effective dose” is meant that the amount sufficient to elicit the desired pharmacological or therapeutic effect, thus resulting in effective prevention or treatment of the condition or disorder. Thus, when treating a CNS disorder, an effective amount of compound is that amount sufficient to pass across the blood-brain barrier of the subject to interact with relevant receptor sites in the brain of the subject. Prevention of the condition or disorder is manifested by delaying the onset of the symptoms of the condition or disorder. Treatment of the condition or disorder is manifested by a decrease in the symptoms associated with the condition or disorder, or an amelioration of the recurrence of the symptoms of the condition of disorder.

The effective dose can vary, depending upon factors such as the condition of the patient, the severity of the symptoms of the disorder, age, weight, metabolic status, concurrent medications, and the manner in which the pharmaceutical composition is administered. Typically, the effective dose of compounds generally requires administering the compound in an amount of about 0.1 to 500 mg/kg of the subject's weight. In an embodiment of the present invention, a dose of about 0.1 to about 300 mg/kg is administered per day indefinitely or until symptoms associated with the condition or disorder cease. Preferably, about 1.0 to 50 mg/kg body weight is administered per day. The required dose is less when administered parenterally.

Those skilled in the art will recognize that the compounds of the present invention may be incorporated with suitable pharmaceutical agents to form a pharmaceutical composition for appropriate administration. Such compositions may limit the active ingredient to a compound of the present invention, or may optionally include other active ingredients or multiple compounds of the present invention.

Pharmaceutical Compositions

The compounds of the present invention are useful in pharmaceutical compositions for systemic administration to mammals including humans as a single agent, or as a primary or adjunct agent with any other medication, chemical, drug or non-drug therapy, or combination thereof. In addition to the compounds, a pharmaceutical composition according to the invention may include one or more pharmaceutical agents including carriers, excipients, actives, fillers, etc.

Administration of the compounds or pharmaceutically acceptable salts or complexes thereof can be employed acutely, or as a single dose, or administered intermittently, or on a regular schedule of unspecified duration, or by continuous infusion of unspecified duration, by an acceptable route of administration including, but not limited to, the oral, buccal, intranasal, pulmonary, transdermal, rectal, vaginal, intradermal, intrathecal, intravenous, intramuscular, and/or subcutaneous routes.

The pharmaceutical preparations can be employed in unit dosage forms, such as tablets, capsules, pills, powders, granules, suppositories, sterile and parenteral solutions, or suspensions, sterile and non-parenteral solutions or suspensions, oral solutions or suspensions, oil in water or water in oil emulsions and the like, containing suitable quantities of an active ingredient. Topical application can be in the form of ointments, creams, lotions, jellies, sprays, douches, and the like. For oral administration either solid or fluid unit dosage forms can be prepared with the compounds of the invention.

Either fluid or solid unit dosage forms can be readily prepared for oral administration. For example, the compounds can be mixed with conventional ingredients such as dicalciumphosphate, magnesium aluminum silicate, magnesium stearate, calcium sulfate, starch, talc, lactose, acacia, methylcellulose and functionally similar materials as pharmaceutical excipients or carriers. A sustained release formulation may optionally be used. Capsules may be formulated by mixing the compound with a pharmaceutical diluent which is inert and inserting this mixture into a hard gelatin capsule having the appropriate size. If soft capsules are desired, a slurry (or other dispersion) of the compound, with an acceptable vegetable, light petroleum or other inert oil can be encapsulated by machine into a gelatin capsule.

Suspensions, syrups, and elixirs may be used for oral administration of fluid unit dosage forms. A fluid preparation including oil may be used for oil soluble forms. A vegetable oil, such as corn oil, peanut oil, or safflower oil, for example, together with flavoring agents, sweeteners, and any preservatives produces an acceptable fluid preparation. A surfactant may be added to water to form syrup for fluid dosages. Hydro-alcoholic pharmaceutical preparations may be used that have an acceptable sweetener, such as sugar, saccharine, or a biological sweetener and a flavoring agent in the form of an elixir.

Pharmaceutical compositions for parental and suppository administration can also be obtained using techniques standard in the art. Another preferred use of these compounds is in a transdermal parenteral pharmaceutical preparation in a mammal such as a human.

The above and other compounds can be present in the reservoir alone, or in combination form with pharmaceutical carriers. The pharmaceutical carriers acceptable for the purpose of this invention are the art known carriers that do not adversely affect the drug, the host, or the material comprising the drug delivery device. Suitable pharmaceutical carriers include sterile water, saline, dextrose, dextrose in water or saline, condensation products of castor oil and ethylene oxide combining about 30 to about 35 moles of ethylene oxide per mole of castor oil, liquid acid, lower alkanols, oils (such as corn oil, peanut oil, sesame oil and the like), with emulsifiers such as mono- or di-glyceride of a fatty acid or a phosphatide (e.g., lecithin and the like), glycols, polyalkyne glycols, aqueous media in the presence of a suspending agent (for example, sodium carboxymethylcellulose), sodium alginate, poly(vinylpyrolidone), and the like (alone or with suitable dispensing agents such as lecithin), or polyoxyethylene stearate and the like. The carrier may also contain adjuvants such as preserving, stabilizing, wetting, emulsifying agents and the like together with the penetration enhancer of this invention.

Although the invention has been described in connection with specific forms thereof, those skilled in the art will appreciate that a wide variety of equivalents may be substituted for the specified elements described herein without departing from the scope and spirit of this invention as described in the claims below.

What is claimed is:

1. A method of inducing analgesia comprising administering to a patient, an analgesia inducing amount of a composition comprising a compound selected from one of Formula I, and Formula II and pharmaceutically acceptable salts thereof: 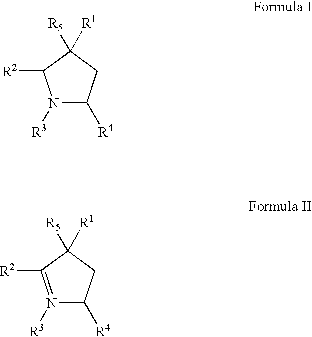
where Formulae I and II include all possible geometric, racemic, diasteriomeric, and enantiomeric forms and where:
R¹ is selected from H, (C₁-C₆)alkyl, (C₃-C₆)cycloalkyl-(C₁-C₆)alkyl, (C₃-C₆)cycloalkyl-(C₁-C₆)alkenyl, aryl, and azaaromatic;
R² is selected from hydrogen, (C₁-C₆)alkyl, (C₂-C₆)alkene, and (C₂-C₆)alkynyl, and in Formula I, R² may also be selected from O═ or HN═;
R³ is selected from hydrogen, (C₁-C₆)alkyl, (C₃-C₆)cycloalkyl, (C₂-C₆) alkenyl, aryl, and aryl(C₁-C₆)alkyl;
R⁴ is selected from (C₁-C₆) alkyl, and (C₃-C₆)cycloalkyl; and
R⁵ is aryl or azaaromatic and may include a bond to R¹ to result in a conjugated ring system.

2. The method of claim 1, wherein R¹ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

3. The method of claim 1, wherein R⁵ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, and aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

4. The method of claim 1 wherein R³ is methyl or ethyl.

5. The method of claim 1, wherein said compound is selected from the following group:

| X | R¹ | R² | R³ | R⁴ | R⁵ | Formula |
|---|---|---|---|---|---|---|
| C | phenyl | CH₂CH₃ | H | CH₃ | phenyl | I |
| C | phenyl | CH₂CH₃ | H | CH₃ | phenyl | I |
| C | phenyl | CH₂CH₃ | CH₃ | CH₃ | phenyl | I |
| C | phenyl | CH2CH₃ | CH₃ | CH₃ | phenyl | I |
| C | phenyl | ═O | H | CH₃ | phenyl | I |
| C | phenyl | ═O | H | CH₃ | phenyl | I |
| C | phenyl | ═O | CH₃ | CH₃ | phenyl | I |
| C | phenyl | ═O | CH₃ | CH₃ | phenyl | I |
| C | phenyl | ═NH | H | CH₃ | phenyl | I |
| C | phenyl | ═NH | H | CH₃ | phenyl | I |
| C | phenyl | ═NCH₃ | H | CH₃ | phenyl | I |
| C | phenyl | ═NCH₃ | H | CH₃ | phenyl | I |
| C | phenyl | —CCH₃CH₂ | H | CH₃ | phenyl | II |
| C | phenyl | —CCH₃CH₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | H | —CH₂CH₃ | H | CH₃ | phenyl | II |
| C | H | —CH₂CH₃ | H | CH₃ | phenyl | II |
| C | H | —CH₂CH₃ | CH₃ | CH₃ | phenyl | II |
| C | H | —CH₂CH₃ | CH₃ | CH₃ | phenyl | II |
| N | H | —CH₂CH₃ | H | CH₃ | 3-pyridinyl | II |
| N | H | —CH₂CH₃ | H | CH₃ | 3-pyridinyl | II |
| N | H | —CH₂CH₃ | CH₃ | CH₃ | 3-pyridinyl | II |
| N | H | —CH₂CH₃ | CH₃ | CH₃ | 3-pyridinyl | II |
| N | H | —CH₂CH₃ | H | CH₃ | 4-chloro-3- | II |
| | | | | | pyridinyl | |
| N | H | —CH₂CH₃ | H | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | phenyl | —CH₂CH₃ | H | CH₃ | pyridinyl | II |
| N | pyridinyl | —CH₂CH₃ | H | CH₃ | pyridinyl | II |
| N | phenyl | —CH₂CH₃ | CH₃ | CH₃ | pyridinyl | II |
| N | pyridinyl | —CH₂CH₃ | CH₃ | CH₃ | pyridinyl | II |
| N | phenyl | —CH₂CH₃ | H | CH₃ | 4-chloro-3- | II |
| | | | | | pyridinyl | |
| N | pyridinyl | —CH₂CH₃ | H | CH₃ | 4-chloro-3- | II |
| | | | | | pyridinyl | |
| N | 4-chloro-3- | —CH₂CH₃ | H | CH₃ | 4-chloro-3- | II |
| | pyridinyl | | | | pyridinyl | |
| N | phenyl | —CH₂CH₃ | CH₃ | CH₃ | 4-chloro-3- | II |
| | | | | | pyridinyl | |
| N | pyridinyl | —CH₂CH₃ | CH₃ | CH₃ | 4-chloro-3- | II |
| | | | | | pyridinyl | |
| N | 4-chloro-3- | —CH₂CH₃ | CH₃ | CH₃ | 4-chloro-3- | II. |
| | pyridinyl | | | | pyridinyl | |

6. The method of claim 1 wherein said analgesia inducing amount of a composition is sufficient to block nicotinic receptors to thereby induce analgesia.

7. A method of deterring abuse of abusive substances comprising administering to a patient, an abuse deterring amount of a composition including compound selected from one of Formula I, and Formula II and pharmaceutically acceptable salts thereof: 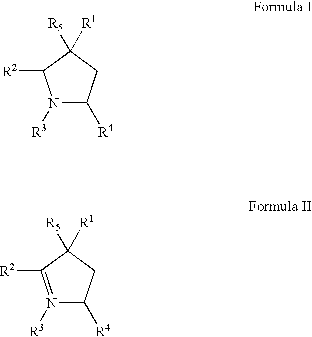
where Formulae I and IT include all possible geometric, racemic, diasteriomeric, and enantiomeric forms and where:
R¹ is selected from H, (C₁-C₆)alkyl, (C₃-C₆)cycloalkyl-(C₁-C₆)alkyl, (C₃-C₆)cycloalkyl-(C₁-C₆)alkenyl, aryl and azaaromatic;
R² is selected from hydrogen, (C₁-C₆)alkyl, (C₂-C₆)alkene, and (C₂-C₆)alkynyl, and in Formula I, R² may additionally be selected from O═ or HN═;
R³ is selected from hydrogen, (C₁₋₆)alkyl, (C₃-C₆)cycloalkyl, (C₂-C₆) alkenyl, aryl, and aryl(C₁-C₆)alkyl;
R⁴ is (C₁-C₆) alkyl, and (C₃-C₆)cycloalkyl; and
R⁵ is aryl or azaaromatic and may include a bond to R¹ to result in a conjugated ring system.

8. The method of claim 7, wherein R¹ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

9. The method of claim 7, wherein R⁵ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, and aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

10. The method of claim 7 wherein R³ is methyl or ethyl.

11. The method of claim 7, wherein said compound is selected from the following group:

| X | R¹ | R² | R³ | R⁴ | R⁵ | Formula |
|---|---|---|---|---|---|---|
| C | phenyl | CH₂CH₃ | H | CH₃ | phenyl | I |
| C | phenyl | CH₂CH₃ | H | CH₃ | phenyl | I |
| C | phenyl | CH₂CH₃ | CH₃ | CH₃ | phenyl | I |
| C | phenyl | CH2CH₃ | CH₃ | CH₃ | phenyl | I |
| C | phenyl | ═O | H | CH₃ | phenyl | I |
| C | phenyl | ═O | H | CH₃ | phenyl | I |
| C | phenyl | ═O | CH₃ | CH₃ | phenyl | I |
| C | phenyl | ═O | CH₃ | CH₃ | phenyl | I |
| C | phenyl | ═NH | H | CH₃ | phenyl | I |
| C | phenyl | ═NH | H | CH₃ | phenyl | I |
| C | phenyl | ═NCH₃ | H | CH₃ | phenyl | I |
| C | phenyl | ═NCH₃ | H | CH₃ | phenyl | I |
| C | phenyl | —CCH₃CH₂ | H | CH₃ | phenyl | II |
| C | phenyl | —CCH₃CH₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | H | —CH₂CH₃ | H | CH₃ | phenyl | II |
| C | H | —CH₂CH₃ | H | CH₃ | phenyl | II |
| C | H | —CH₂CH₃ | CH₃ | CH₃ | phenyl | II |
| C | H | —CH₂CH₃ | CH₃ | CH₃ | phenyl | II |
| N | H | —CH₂CH₃ | H | CH₃ | 3-pyridinyl | II |
| N | H | —CH₂CH₃ | H | CH₃ | 3-pyridinyl | II |
| N | H | —CH₂CH₃ | CH₃ | CH₃ | 3-pyridinyl | II |
| N | H | —CH₂CH₃ | CH₃ | CH₃ | 3-pyridinyl | II |
| N | H | —CH₂CH₃ | H | CH₃ | 4-chloro-3- | II |
| | | | | | pyridinyl | |
| N | H | —CH₂CH₃ | H | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | phenyl | —CH₂CH₃ | H | CH₃ | pyridinyl | II |
| N | pyridinyl | —CH₂CH₃ | H | CH₃ | pyridinyl | II |
| N | phenyl | —CH₂CH₃ | CH₃ | CH₃ | pyridinyl | II |
| N | pyridinyl | —CH₂CH₃ | CH₃ | CH₃ | pyridinyl | II |
| N | phenyl | —CH₂CH₃ | H | CH₃ | 4-chloro-3- | II |
| | | | | | pyridinyl | |
| N | pyridinyl | —CH₂CH₃ | H | CH₃ | 4-chloro-3- | II |
| | | | | | pyridinyl | |
| N | 4-chloro-3- | —CH₂CH₃ | H | CH₃ | 4-chloro-3- | II |
| | pyridinyl | | | | pyridinyl | |
| N | phenyl | —CH₂CH₃ | CH₃ | CH₃ | 4-chloro-3- | II |
| | | | | | pyridinyl | |
| N | pyridinyl | —CH₂CH₃ | CH₃ | CH₃ | 4-chloro-3- | II |
| | | | | | pyridinyl | |
| N | 4-chloro-3- | —CH₂CH₃ | CH₃ | CH₃ | 4-chloro-3- | II. |
| | pyridinyl | | | | pyridinyl | |

12. The method of claim 7 wherein said amount of compound selected from one of Formula I, and Formula II and pharmaceutically acceptable salts is sufficient to block nicotinic receptors to thereby deter abuse of abusive substances.

13. A compound of selected from the group consisting of Formula I, Formula II, and pharmaceutically acceptable salts thereof: 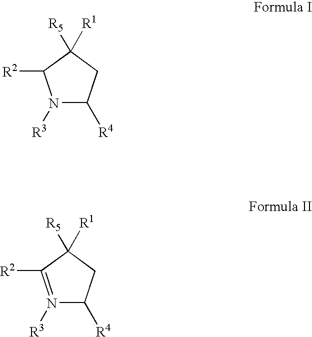
where Formulae I and II include all possible geometric, racemic, diasteriomeric, and enantiomeric forms and where:
R¹ is selected from H, (C₁-C₆)alkyl, (C₃-C₆)cycloalkyl-(C₁-C₆)alkyl, (C₃-C₆)cycloalkyl-(C₁-C₆)alkenyl, aryl and azaaromatic;
R¹ is selected from hydrogen, (C₁-C₆)alkyl, (C₂-C₆)alkene, and (C₂-C₆)alkynyl, and in Formula I, R² may additionally be selected from O═ or HN═;
R³ is selected from hydrogen, (C₁-C₆)alkyl, (C₃-C₆)cycloalkyl, C₂-C₆ alkenyl, aryl, and aryl(C₁-C₆)alkyl;
R⁴ is C₁-C₆ alkyl, and (C₃-C₆)cycloalkyl; and
R⁵ is aryl or azaaromatic and may form a bond to R¹ to result in a conjugated ring system, except compounds of Formula II where R⁵═R¹═ phenyl, R₂ is ethyl, R⁴ is H, and R₃ is H or CH₃.

14. The compound of 13, wherein R¹ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

15. The compound of claim 13, wherein R⁵ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

16. The compound of claim 13 wherein R³ is methyl or ethyl.

17. The compound of claim 13, wherein said compound is selected from the following group:

| X | R¹ | R² | R³ | R⁴ | R⁵ | Formula |
|---|---|---|---|---|---|---|
| C | phenyl | CH₂CH₃ | H | CH₃ | phenyl | I |
| C | phenyl | CH₂CH₃ | H | CH₃ | phenyl | I |
| C | phenyl | CH₂CH₃ | CH₃ | CH₃ | phenyl | I |
| C | phenyl | CH2CH₃ | CH₃ | CH₃ | phenyl | I |
| C | phenyl | ═O | H | CH₃ | phenyl | I |
| C | phenyl | ═O | H | CH₃ | phenyl | I |
| C | phenyl | ═O | CH₃ | CH₃ | phenyl | I |
| C | phenyl | ═O | CH₃ | CH₃ | phenyl | I |
| C | phenyl | ═NH | H | CH₃ | phenyl | I |
| C | phenyl | ═NH | H | CH₃ | phenyl | I |
| C | phenyl | ═NCH₃ | H | CH₃ | phenyl | I |
| C | phenyl | ═NCH₃ | H | CH₃ | phenyl | I |
| C | phenyl | —CCH₃CH₂ | H | CH₃ | phenyl | II |
| C | phenyl | —CCH₃CH₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | H | —CH₂CH₃ | H | CH₃ | phenyl | II |
| C | H | —CH₂CH₃ | H | CH₃ | phenyl | II |
| C | H | —CH₂CH₃ | CH₃ | CH₃ | phenyl | II |
| C | H | —CH₂CH₃ | CH₃ | CH₃ | phenyl | II |
| N | H | —CH₂CH₃ | H | CH₃ | 3-pyridinyl | II |
| N | H | —CH₂CH₃ | H | CH₃ | 3-pyridinyl | II |
| N | H | —CH₂CH₃ | CH₃ | CH₃ | 3-pyridinyl | II |
| N | H | —CH₂CH₃ | CH₃ | CH₃ | 3-pyridinyl | II |
| N | H | —CH₂CH₃ | H | CH₃ | 4-chloro-3- | II |
| | | | | | pyridinyl | |
| N | H | —CH₂CH₃ | H | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | phenyl | —CH₂CH₃ | H | CH₃ | pyridinyl | II |
| N | pyridinyl | —CH₂CH₃ | H | CH₃ | pyridinyl | II |
| N | phenyl | —CH₂CH₃ | CH₃ | CH₃ | pyridinyl | II |
| N | pyridinyl | —CH₂CH₃ | CH₃ | CH₃ | pyridinyl | II |
| N | phenyl | —CH₂CH₃ | H | CH₃ | 4-chloro-3- | II |
| | | | | | pyridinyl | |
| N | pyridinyl | —CH₂CH₃ | H | CH₃ | 4-chloro-3- | II |
| | | | | | pyridinyl | |
| N | 4-chloro-3- | —CH₂CH₃ | H | CH₃ | 4-chloro-3- | II |
| | pyridinyl | | | | pyridinyl | |
| N | phenyl | —CH₂CH₃ | CH₃ | CH₃ | 4-chloro-3- | II |
| | | | | | pyridinyl | |
| N | pyridinyl | —CH₂CH₃ | CH₃ | CH₃ | 4-chloro-3- | II |
| | | | | | pyridinyl | |
| N | 4-chloro-3- | —CH₂CH₃ | CH₃ | CH₃ | 4-chloro-3- | II. |
| | pyridinyl | | | | pyridinyl | |

18. The compound according to claim 13, wherein said analogs are in the form of pharmaceutically acceptable salts.

19. The compound of claim 18, wherein said pharmaceutically acceptable salts are inorganic acid addition salts, organic acid addition salts, salts with acidic amino acids, and hydrates or solvates thereof with alcohols and other solvents.

20. The compound of claim 19, wherein said analog is an inorganic acid addition salt selected from the group consisting of hydrochloride, hydrobromide, sulfate, phosphate and nitrate.

21. The compound of claim 19, wherein said analog is an organic acid addition salts salt selected from the group consisting of acetate, galactarate, propionate, succinate, lactate, glycolate, malate, tartrate, citrate, maleate, fumarate, methanesulfonate, salicylate, p-toluenesulfonate, benzenesulfonate, and ascorbate.

22. The compound of claim 19, wherein said analog is a salt with acidic amino acids selected from the group consisting of aspartate and glutamate.

23. A pharmaceutical Composition comprising:
a pharmaceutically acceptable agents; and
a compound selected from one of Formula I and Formula II, and pharmaceutically acceptable salts thereof: 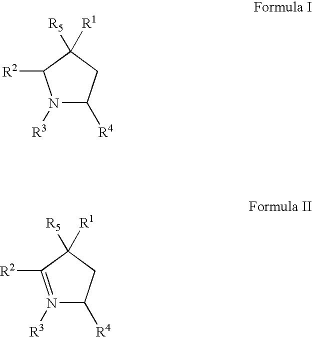
where Formulae I and II include all possible geometric, racemic, diasteriomeric, and enantiomeric forms and where:
R¹ is selected from H, (C₁-C₆)alkyl, (C₃-C₆)cycloalkyl-(C₁-C₆)alkyl, (C₃-C₆)cycloalkyl-(C₁-C₆)alkenyl, aryl and azaaromatic;
R² is selected from hydrogen, (C₁-C₆)alkyl, (C₂-C₆)alkene, and (C₂-C₆)alkynyl, and in Formula I, R² may additionally be selected from O═ or HN═;
R³ is selected from hydrogen, (C₁-C₆)alkyl, (C₃-C₆)cycloalkyl, (C₂-C₆) alkenyl, aryl, and aryl(C₁-C₆)alkyl;
R⁴ is (C₁-C₆) alkyl, and (C₃-C₆)cycloalkyl; and R⁵ is aryl or azaaromatic and may form a bond to R¹ to result in a conjugated ring system; and
wherein said amount is sufficient to induce analgesia and/or deter abuse of abusive substances.

24. The composition of claim 23, wherein R¹ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

25. The composition of claim 23, wherein R⁵ is selected from the group consisting of aryl and azaaromatic, each having 1-5 substituents independently selected from the group consisting of hydrogen, (C₁-C₆)alkyl, (C₃-C₆)cycloalkyl, (C₂-C₆)alkenyl, aryl, aryl(C₁-C₆)alkyl, N-methylamino, N,N-dimethylamino, carboxylate, (C₁-C₃)alkylcarboxylate, carboxaldehyde, acetoxy, propionyloxy, isopropionyloxy, cyano, aminomethyl, N-methylaminomethyl, N,N-dimethylaminomethyl, carboxamide, N-methylcarboxamide, N,N-dimethylcarboxamide, acetyl, propionyl, formyl, benzoyl, sulfate, methylsulfate, hydroxyl, methoxy, ethoxy, propoxy, isopropoxy, thiol, methylthio, ethylthio, propiothiol, fluoro, chloro, bromo, iodo, trifluoromethyl, propargyl, nitro, carbamoyl, ureido, azido, isocyanate, thioisocyanate, hydroxylamino, and nitroso.

26. The composition of claim 23 wherein R³ is methyl or ethyl.

27. The composition of claim 23, wherein said compound is selected from the following group:

| X | R¹ | R² | R³ | R⁴ | R⁵ | Formula |
|---|---|---|---|---|---|---|
| C | phenyl | CH₂CH₃ | H | CH₃ | phenyl | I |
| C | phenyl | CH₂CH₃ | H | CH₃ | phenyl | I |
| C | phenyl | CH₂CH₃ | CH₃ | CH₃ | phenyl | I |
| C | phenyl | CH2CH₃ | CH₃ | CH₃ | phenyl | I |
| C | phenyl | ═O | H | CH₃ | phenyl | I |
| C | phenyl | ═O | H | CH₃ | phenyl | I |
| C | phenyl | ═O | CH₃ | CH₃ | phenyl | I |
| C | phenyl | ═O | CH₃ | CH₃ | phenyl | I |
| C | phenyl | ═NH | H | CH₃ | phenyl | I |
| C | phenyl | ═NH | H | CH₃ | phenyl | I |
| C | phenyl | ═NCH₃ | H | CH₃ | phenyl | I |
| C | phenyl | ═NCH₃ | H | CH₃ | phenyl | I |
| C | phenyl | —CCH₃CH₂ | H | CH₃ | phenyl | II |
| C | phenyl | —CCH₃CH₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | phenyl | —CH(CH₃)₂ | H | CH₃ | phenyl | II |
| C | phenyl | —CH(CH₃)₂ | CH₃ | CH₃ | phenyl | II |
| C | H | —CH₂CH₃ | H | CH₃ | phenyl | II |
| C | H | —CH₂CH₃ | H | CH₃ | phenyl | II |
| C | H | —CH₂CH₃ | CH₃ | CH₃ | phenyl | II |
| C | H | —CH₂CH₃ | CH₃ | CH₃ | phenyl | II |
| N | H | —CH₂CH₃ | H | CH₃ | 3-pyridinyl | II |
| N | H | —CH₂CH₃ | H | CH₃ | 3-pyridinyl | II |
| N | H | —CH₂CH₃ | CH₃ | CH₃ | 3-pyridinyl | II |
| N | H | —CH₂CH₃ | CH₃ | CH₃ | 3-pyridinyl | II |
| N | H | —CH₂CH₃ | H | CH₃ | 4-chloro-3- | II |
| | | | | | pyridinyl | |
| N | H | —CH₂CH₃ | H | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | H | —CH₂CH₃ | CH₃ | CH₃ | 4-chloro-3- | II |
| | | | | | pyridinyl | |
| N | phenyl | —CH₂CH₃ | H | CH₃ | pyridinyl | II |
| N | pyridinyl | —CH₂CH₃ | H | CH₃ | pyridinyl | II |
| N | phenyl | —CH₂CH₃ | CH₃ | CH₃ | pyridinyl | II |
| N | pyridinyl | —CH₂CH₃ | CH₃ | CH₃ | pyridinyl | II |
| N | phenyl | —CH₂CH₃ | H | CH₃ | 4-chloro-3- | II |
| | | | | | pyridinyl | |
| N | pyridinyl | —CH₂CH₃ | H | CH₃ | 4-chloro-3- | II |
| | | | | | pyridinyl | |
| N | 4-chlora-3- | —CH₂CH₃ | H | CH₃ | 4-chloro-3- | II |
| | pyridinyl | | | | pyridinyl | |
| N | phenyl | —CH₂CH₃ | CH₃ | CH₃ | 4-chloro-3- | II |
| | | | | | pyridinyl | |
| N | pyridinyl | —CH₂CH₃ | CH₃ | CH₃ | 4-chloro-3- | II |
| | | | | | pyridinyl | |
| N | 4-chloro-3- | —CH₂CH₃ | CH₃ | CH₃ | 4-chloro-3- | II. |
| | pyridinyl | | | | pyridinyl | |

28. The pharmaceutical composition according to claim 23, wherein said analogs are in the form of pharmaceutically acceptable salts.

29. The pharmaceutical composition of claim 28, wherein said pharmaceutically acceptable salts are inorganic acid addition salts, organic acid addition salts, salts with acidic amino acids, and hydrates or solvates thereof with alcohols and other solvents.

30. The pharmaceutical composition of claim 29, wherein said analog is an inorganic acid addition salt selected from the group consisting of hydrochloride, hydrobromide, sulfate, phosphate and nitrate.

31. The pharmaceutical composition of claim 29, wherein said analog is an organic acid addition salts salt selected from the group consisting of acetate, galactarate, propionate, succinate, lactate, glycolate, malate, tartrate, citrate, maleate, fumarate, methanesulfonate, salicylate, p-toluenesulfonate, benzenesulfonate, and ascorbate.

32. The pharmaceutical composition of claim 29, wherein said analog is a salt with acidic amino acids selected from the group consisting of aspartate and glutamate.
